(19) United States
(12) Patent Application Publication
(10) Pub. No.: US 2013/0046353 A1
(43) Pub. Date: Feb. 21, 2013

(54) OVAL TUNNEL DILATORS

(71) Applicants: Gary Robert McCarthy, East Bridgewater, MA (US); Thomas Wayne Hughes, East Walpole, MA (US); Bradley Allen Waterson, Northbridge, MA (US); Andrew Francis Rubino, Westport, MA (US); Roger Raymond Cassidy, Pepperell, MA (US)

(72) Inventors: Gary Robert McCarthy, East Bridgewater, MA (US); Thomas Wayne Hughes, East Walpole, MA (US); Bradley Allen Waterson, Northbridge, MA (US); Andrew Francis Rubino, Westport, MA (US); Roger Raymond Cassidy, Pepperell, MA (US)

(73) Assignee: SMITH & NEPHEW, INC., Andover, MA (US)

(21) Appl. No.: 13/212,018

(22) Filed: Aug. 17, 2011

(51) Int. Cl. A61B 17/56; A61M 29/00

(52) U.S. Cl. 606/86 R; 606/191

(57) ABSTRACT

A dilator apparatus having a body having a proximal end and a distal end and a longitudinal axis defined therethrough, and a dilation member coupled to the distal end of the body, the dilation member having a dilation body and a first distal nose coupled to the dilation body. Further, the width of the dilation body is larger than a width of the first distal nose.

### BACKGROUND OF INVENTION

Anterior cruciate ligament (ACL) reconstruction techniques are applied to an increased number of reconstruction operations. Drilling bone tunnels, e.g. femoral tunnels, in ACL reconstruction procedures is also increasing, as the number of ACL reconstruction operations has increased.

Accordingly, there exists a need for a dilator apparatus for effectively dilating a hole formed in a bone within a patient's body.

### SUMMARY OF INVENTION

According to one aspect of the present invention, there is provided a dilator apparatus having a proximal end and a distal end and a longitudinal axis defined therethrough, and a dilation member coupled to the distal end of the body, the dilation member having a dilation body and a first distal nose coupled to the dilation body, in which a width of the dilation body is larger than a width of the first distal nose.

According to another aspect of the present invention, there is provided a method for dilating a bone, the method comprising forming a first hole within a bone, inserting a dilator apparatus into the first hole, the dilator apparatus comprising a body and a dilation member, in which the dilation member is coupled to a distal end of the body, and advancing the dilator apparatus within the first hole formed in the bone to a predetermined depth within the bone.

### BRIEF DESCRIPTION OF DRAWINGS

FIG. 1A is a perspective view of a dilator apparatus in accordance with embodiments disclosed herein.

FIG. 1B is a close-up view of a distal end of the dilator apparatus of FIG. 1B.

FIG. 2A is a perspective view of another embodiment of a dilator apparatus in accordance with embodiments disclosed herein.

FIG. 2B is a close-up view of a distal end of the dilator apparatus of FIG. 2B.

### DETAILED DESCRIPTION

The following is directed to various exemplary embodiments of the disclosure. Although one or more of these embodiments may be preferred, the embodiments disclosed should not be interpreted, or otherwise used, as limiting the scope of the disclosure, including the claims. In addition, those having ordinary skill in the art will appreciate that the following description has broad application, and the discussion of any embodiment is meant only to be exemplary of that embodiment, and not intended to suggest that the scope of the disclosure, including the claims, is limited to that embodiment.

Certain terms are used throughout the following description and claims refer to particular features or components. As those having ordinary skill in the art will appreciate, different persons may refer to the same feature or component by different names. This document does not intend to distinguish between components or features that differ in name but not function. The figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first component is coupled to a second component, that connection may be through a direct connection, or through an indirect connection via other components, devices, and connections. Further, the terms “axial” and “axially” generally mean along or substantially parallel to a central or longitudinal axis, while the terms “radial” and “radially” generally mean perpendicular to a central, longitudinal axis.

Additionally, directional terms, such as “above,” “below,” “upper,” “lower,” etc., are used for convenience in referring to the accompanying drawings. In general, “above,” “upper,” “upward,” and similar terms refer to a direction toward a proximal end of an instrument, device, apparatus, or system, and “below,” “lower,” “downward,” and similar terms refer to a direction toward a distal end of an instrument, device, apparatus, or system, but is meant for illustrative purposes only, and the terms are not meant to limit the disclosure.

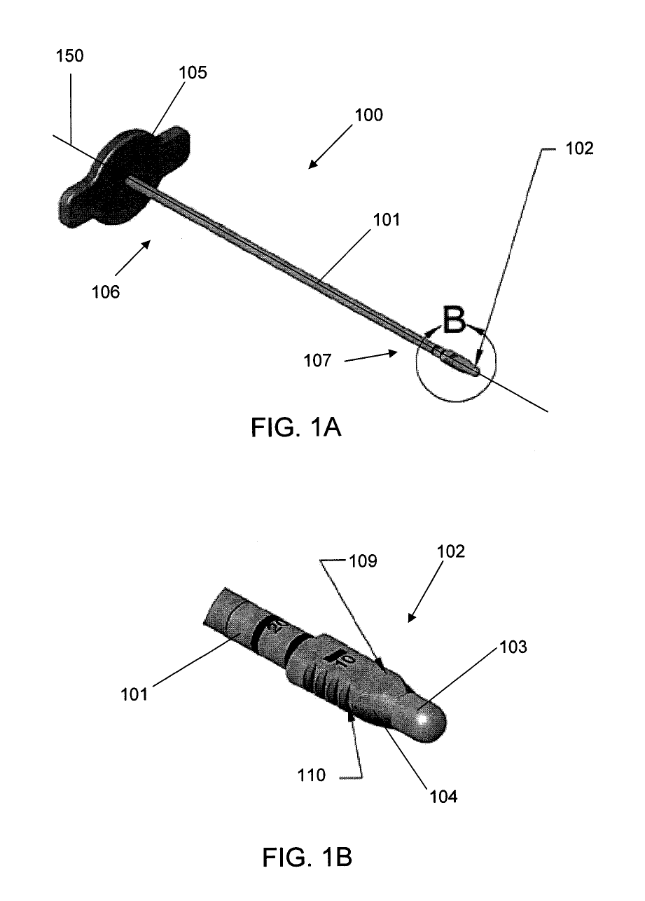

Referring to FIGS. 1A and 1B, multiple views of a dilator apparatus 100, according to embodiments disclosed herein, are shown. In one or more embodiments, the dilator apparatus 100 may include a body 101 having a proximal end 106 and a distal end 107 and a longitudinal axis 150 defined therethrough. In one or more embodiments, the dilator apparatus 100 may include a dilation member 102 coupled to the distal end 107 of the body 101, the dilation member 102 having a dilation body 109 and a first distal nose 103 coupled to the dilation body 109, in which a width of the dilation body 109 is larger than a width of the first distal nose 103.

In one or more embodiments, the dilator apparatus 100 may include a handle 105 coupled to the proximal end 106 of the body 101. As shown in FIG. 1A, the handle 105 is secured to the proximal end 106 of the body 101 and may provide a gripping surface on the dilator apparatus 100 for a surgeon. Those having ordinary skill in the art will appreciate that the handle 105 may be any shape and size. For example, in one or more embodiments, the handle 105 of the dilator apparatus 100 may be rod-shaped, extending substantially 90 degrees from the proximal end 106 of the body 101, i.e., along a plane that is substantially orthogonal to the longitudinal axis 150 of the body 101. Alternatively, in one or more embodiments, the handle may be triangular in shape and may not necessarily extend 90 degrees from the proximal end 106 of the body 101. For example, a 120 degree angle may be formed between the longitudinal axis 150 of the body 101 and the handle 105.

In one or more embodiments, the width of the dilation body 109 may be larger than a width of the body 101. As shown in FIGS. 1A and 1B, the width of the first distal nose 103 of the dilation member 102 may be smaller than the dilation body 109 of the dilation member 102. Further, as shown in FIGS. 1A and 1B, the width of the body 101 may be smaller than the dilation body 109 of the dilation member 102. However, those having ordinary skill in the art will appreciate that, in one or more embodiments, the width of the body 101 may be equal to, or larger than, the dilation body 109 of the dilation member 102. For example, in one or more embodiments, the width of the first distal nose 103 may be smaller than both the width of the dilation body 109 of the dilation member 102 as well as the width of the body 101, in which the width of the dilation body 109 of the dilation member 102 may be smaller than the width of the body 101. In other words, in one or more embodiments, the width of the body 101 may be larger than both the width of the dilation body 109 of the dilation member 102 as well as the width of the first distal nose 103, in which the width of the dilation body 109 of the dilation member 102 may be larger than the width of the first distal nose 103.

In one or more embodiments, a region between the first distal nose 103 and the dilation body 109 may be a tapered region 104. As shown in FIG. 1B, and as discussed above, the width of the dilation body 109 of the dilation member 102 may be larger than the width of the first distal nose 103. Further, as shown, the region between the first distal nose 103 and the dilation body 109 of the dilation member 102 is a tapered region 104. In one or more embodiments, the tapered region 104 may provide a smooth transition between the width of the first distal nose 103 and the width of the dilation body 109 of the dilation member 102. Those having ordinary skill in the art will appreciate that the tapered region 104 may be concave, convex, or a straight edge formed between the first distal nose 103 and the dilation body 109 of the dilation member 102. For example, in one or more embodiments, the tapered region 104 may be a concave, smooth surface formed between the first distal nose 103 and the dilation body 109 of the dilation member 102. Alternatively, in one or more embodiments, the tapered region 104 may be either a straight or a convex, smooth surface formed between the first distal nose 103 and the dilation body 109 of the dilation member 102. The tapered region 104 may provide a smooth surface between the first distal nose 103 and the dilation body 109 of the dilation member 102 that may assist with insertion and removal of the dilation apparatus 100 into/out of a bone (not shown).

In one or more embodiments, a cross-section of the dilation body 109 of the dilation member 102 may be substantially oval-shaped. However, those having ordinary skill in the art will appreciate that the cross-section of the dilation body 109 of the dilation member 102 may be any shape known in the art. For example, the cross-section of the dilation body 109 of the dilation member 102 may be circular, elliptical, hexagonal, triangular, or any other shape known in the art. In one or more embodiments, the major axis of the cross-section of the dilation member 102, i.e., the largest diameter of the cross-section of the dilation member 102, may be 6 mm, 6.5 mm, 7 mm, or 7.5 mm. As such, graft sizes from 6 mm, 6.5 mm, 7 mm, or 7.5 mm may be inserted into the dilated hole formed by the dilator assembly 100. However, those having ordinary skill in the art will appreciate that the major axis of the cross-section of the dilation member 102 may be any size and may accommodate any graft size that may be inserted into the body.

Further, in one or more embodiments, the first distal nose 103 may be approximately 4.5 mm in diameter. However, those having ordinary skill in the art will appreciate that the first distal nose 103 may have a diameter of any size and is not limited to a constant diameter of 4.5 mm. For example, in one or more embodiments, the diameter of the first distal nose 103 may be larger or smaller than 4.5 mm. Further, those having ordinary skill in the art will appreciate that the diameter of the first distal nose 103 may not be constant throughout. For example, the first distal nose 103 may have a diameter that increases, or decreases, along the longitudinal axis 150. Further, in one or more embodiments, the cross-section of the first distal nose 103 may be circular. However, in one or more embodiments, the cross-section of the first distal nose 103 may be elliptical, square, rectangular, hexagonal, or any other shape known in the art. In one or more embodiments, the first distal nose 103 may assist a surgeon when inserting the dilator apparatus 100 into a hole (not shown) formed in a bone (not shown).

Further, in one or more embodiments, the dilation body 109 of the dilation member 102 may include a plurality of broaches 110. As shown in FIG. 1B, the plurality of broaches 110 are formed on an outer surface of the dilation body 109 of the dilation member 102. As shown in FIG. 1B, the plurality of broaches 110 may be a plurality of ridges formed on the outer surface of the dilation body 109 of the dilation member 102. However, those having ordinary skill in the art will appreciate that the plurality of broaches 110 may be any formation or plurality of apertures that may form a textured surface on the outer surface of the dilation body 109 of the dilation member 102. For example, in one or more embodiments, the plurality of broaches 110 may be a series, or grid, of bumps or formations that may form a textured surface on the outer surface of the dilation body 109 of the dilation member 102. Although the plurality of broaches 110 are shown formed on opposite surfaces of the dilation body 109 of the dilation member 102, above the tapered region 104, those having ordinary skill in the art will appreciate that the plurality of broaches 110 may be formed anywhere on the dilator apparatus 100. For example, in one or more embodiments, the plurality of broaches 110 may be formed on the entire outer surface of the dilation member 102. The plurality of broaches 110 may assist with bone removal during insertion of the dilator apparatus 100 into a bone (not shown).

In one or more embodiments, the dilator apparatus 100 may be formed from any substantially rigid material known in the art. For example, the dilator apparatus 100 may be formed from steel, ceramic, plastic, polymer, or any combination thereof, known in the art.

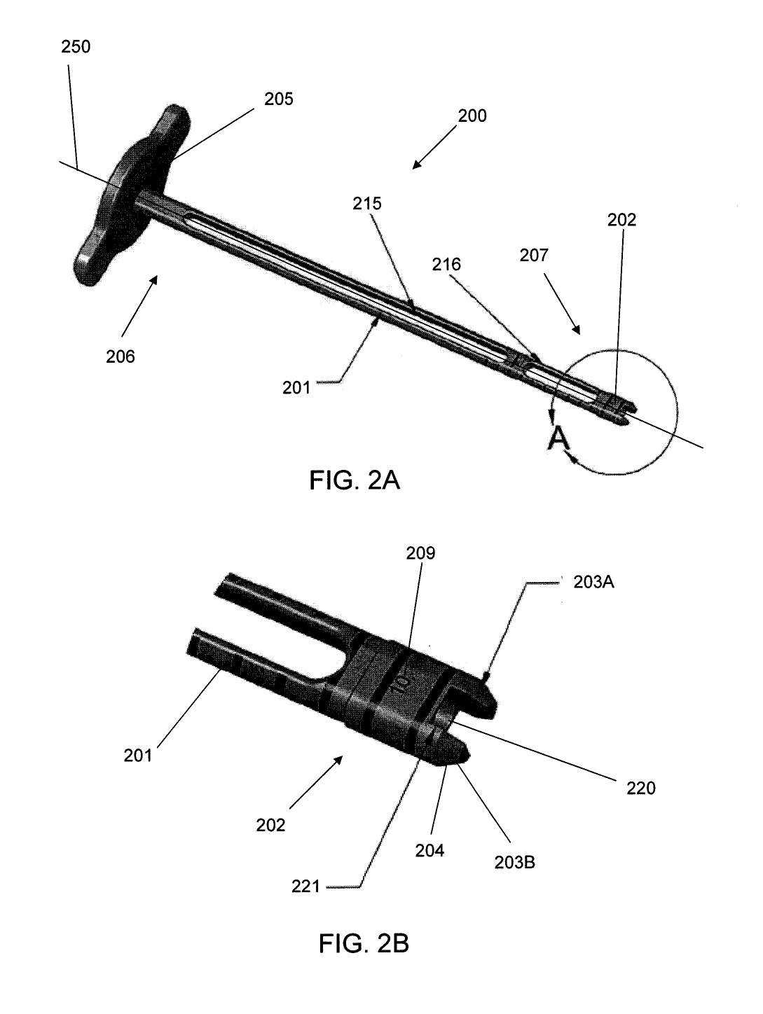

Referring now to FIGS. 2A and 2B, multiple views of a dilator apparatus 200, according to embodiments disclosed herein, are shown. In one or more embodiments, the dilator apparatus 200 may include a body 201 having a proximal end 206 and a distal end 207 and a longitudinal axis 250 defined therethrough. In one or more embodiments, the dilator apparatus 200 may include a dilation member 202 coupled to the distal end 207 of the body 201, the dilation member 202 having a dilation body 209 and a first distal nose 203A coupled to the dilation body 209 and a second distal nose 203B coupled to the dilation body 209, in which a width of the dilation body 209 is larger than both a width of the first distal nose 203A and a width of the second distal nose 203B.

In one or more embodiments, the dilator apparatus 200 may include a handle 205 coupled to the proximal end 206 of the body 201. As shown in FIG. 2A, the handle 205 is secured to the proximal end 206 of the body 201 and may provide a gripping surface on the dilator apparatus 200 for a surgeon. As discussed above, those having ordinary skill in the art will appreciate that the handle 205 may be any shape and size. For example, in one or more embodiments, the handle 205 of the dilator apparatus 200 may be rod-shaped, extending substantially 90 degrees from the proximal end 206 of the body 201, i.e., along a plane that is substantially orthogonal to the longitudinal axis 250 of the body 201. Alternatively, in one or more embodiments, the handle may be triangular in shape and may not necessarily extend 90 degrees from the proximal end 206 of the body 201. For example, a 120 degree angle may be formed between the longitudinal axis 250 of the body 201 and the handle 205.

In one or more embodiments, the width of the dilation body 209 may be larger than a width of the body 201. As shown in FIGS. 2A and 2B, the width of the first distal nose 203A and the width of the second distal nose 203B of the dilation member 202 may be smaller than the dilation body 209 of the dilation member 202. Further, as shown, the width of the body 201 may be smaller than the dilation body 209 of the dilation member 202. However, as discussed above, those having ordinary skill in the art will appreciate that, in one or more embodiments, the width of the body 201 may be equal to, or larger than, the dilation body 209 of the dilation member 202. For example, in one or more embodiments, both the width of the first distal nose 203A and the width of the second distal nose 203B may be smaller than both the width of the dilation body 209 of the dilation member 202 as well as the width of the body 201, in which the width of the dilation body 209 of the dilation member 202 may be smaller than the width of the body 201. In other words, in one or more embodiments, the width of the body 201 may be larger than both the width of the dilation body 209 of the dilation member 202 as well as both the width of the first distal nose 203A and the width of second distal nose 203B, in which the width of the dilation body 209 of the dilation member 202 may be larger than both the width of the first distal nose 203A and the width of the second distal nose 203B.

In one or more embodiments, a region between the first distal nose 203A and the second distal nose 203B and the dilation body 109 may be a tapered region 204. As shown in FIG. 2B, and as discussed above, the width of the dilation body 209 of the dilation member 202 may be larger than both the width of the first distal nose 203A and the width of the second distal nose 203B. Further, as shown, the region between the distal noses 203A, 203B and the dilation body 209 of the dilation member 202 is a tapered region 204. As discussed above, in one or more embodiments, the tapered region 204 may provide a smooth transition between the widths of the distal noses 203A, 203B and the width of the dilation body 209 of the dilation member 202. Those having ordinary skill in the art will appreciate that the tapered region 204 may be concave, convex, or a straight edge formed between the distal noses 203A, 203B and the dilation body 209 of the dilation member 202. For example, in one or more embodiments, the tapered region 204 may be a concave, smooth surface formed between the distal noses 203A, 203B and the dilation body 209 of the dilation member 202. Alternatively, in one or more embodiments, the tapered region 204 may be either a straight or a convex, smooth surface formed between the distal noses 203A, 203B and the dilation body 209 of the dilation member 202.

In one or more embodiments, a cross-section of the dilation body 209 of the dilation member 202 may be substantially oval-shaped. However, as discussed above, those having ordinary skill in the art will appreciate that the cross-section of the dilation body 209 of the dilation member 202 may be any shape known in the art. For example, the cross-section of the dilation body 209 of the dilation member 202 may be circular, elliptical, hexagonal, triangular, or any other shape known in the art. In one or more embodiments, the major axis of the cross-section of the dilation member 202, i.e., the largest diameter of the cross-section of the dilation member 202, may be 8 mm, 8.5 mm, 9 mm, 9.5 mm, or 10 mm. As such, graft sizes from 8 mm, 8.5 mm, 9 mm, 9.5 mm, or 10 mm may be inserted into the dilated hole formed by the dilator assembly 200. However, those having ordinary skill in the art will appreciate that the major axis of the cross-section of the dilation member 202 may be any size and may accommodate any graft size that may be inserted into the body.

Further, in one or more embodiments, a cross-section of the body 201 may be substantially oval-shaped. However, those having ordinary skill in the art will appreciate that the cross-section of the dilation body 209 of the dilation member 202 may be any shape known in the art. For example, the cross-section of the dilation body 209 of the dilation member 202 may be circular, elliptical, hexagonal, triangular, or any other shape known in the art.

In one or more embodiments, at least one window may be formed through the body. As shown in FIG. 2A, windows 215, 216 are formed through the body 201 of the dilator apparatus 200. The windows 215, 216 may reduce the weight of the body 201 of the dilator apparatus 200 and may provide a surgeon increased vision during a procedure. Although two windows 215, 216 are shown formed through the body 201 of the dilator apparatus 200, those having ordinary skill in the art will appreciate that one or more windows may be formed through the body 201 of the dilator apparatus 200. For example, one, three, four, or more windows may be formed through the body 201 of the dilator apparatus 200.

Further, in one or more embodiments, a hole may be formed through the body and the dilation member along the longitudinal axis of the body. As shown in FIG. 2B, a hole 220 is formed through the body 201 and the dilation member 202 along the longitudinal axis 250 of the body 201. In one or more embodiments, the hole 220 may be formed through the dilation member 202, the entire length of the body 201, as well as through the handle 205. The hole 220 may be configured to receive a guide pin (not shown), which may assist in guiding the dilator apparatus 200 into a bone (not shown). Furthermore, in one or more embodiments, a notch 221 may be formed in the dilation member 202, between the first distal nose 203A and the second distal nose 203B. In one or more embodiments, the notch 221 may allow the distal noses 203A, 203B to be more easily situated within holes (not shown) formed in a bone (not shown).

Although not shown, in one or more embodiments, the dilator apparatus 200 may include a plurality of broaches, e.g., the plurality of broaches 110 discussed above, formed on an outer surface of the dilation member 202. Further, in one or more embodiments, the dilator apparatus 200 may be formed from any substantially rigid material known in the art. For example, the dilator apparatus 200 may be formed from steel, ceramic, plastic, polymer, or any combination thereof, known in the art.

A method for dilating a bone, in accordance with embodiments disclosed herein, may include forming a first hole within a bone, inserting a dilator apparatus into the first hole, the dilator apparatus having a body and a dilation member, in which the dilation member is coupled to a distal end of the body, and advancing the dilator apparatus within the first hole formed in the bone to a predetermined depth within the bone.

For example, referring to FIGS. 1A and 1B, a first hole (not shown) may be formed within a bone (not shown), and the dilator apparatus 100, e.g., the first distal nose 103 of the dilator apparatus 100, may be inserted into the first hole. The first hole may be formed by a drill, by inserting a guide pin (not shown) into the bone, or by any other method known in the art. Further, the dilator apparatus 100 may be advanced into the first hole formed in the bone to a predetermined depth within the bone. As discussed above, the width of the dilation body 109 of the dilation member 102 may be larger than the width of the first distal nose 103. Accordingly, in one or more embodiments, a diameter of the first hole may increase as the dilator apparatus 100 is advanced into the first hole beyond the first distal nose 103. In other words, the first hole formed in the bone may be dilated as the dilator apparatus 100 is advanced into the first hole beyond the first distal nose 103. In one or more embodiments, advancing the dilator apparatus 100 within the first hole formed in the bone may include advancing the dilator apparatus 100 within the first hole formed in the bone using a mallet or hammer. However, those having ordinary skill in the art will appreciate that any other method known in the art may be used to advance the dilator apparatus 100 within the first hole formed in the bone. For example, the dilator apparatus 100 may be pushed, or forced, into the first hole by any means known in the art. As the dilator apparatus 100 is advanced into the bone, portions of the bone may be removed by the dilator apparatus 100. For example, portions of the bone may be removed by the dilation body 109 of the dilator apparatus 100, as the width of the dilation body 109 may be larger than a width of the first distal nose 103, as discussed above. Further, the plurality of broaches 110 may assist with the removal of portions of the bone as the dilator assembly 100 is advanced into the bone.

The method may also include inserting a first passing pin through the bone, measuring a size of the first hole and the predetermined depth within the bone with a depth probe, and orienting the dilator apparatus within the first hole formed in the bone.

For example, a surgeon may insert a first passing pin through a bone, e.g. a femur, and may drill a first hole into the bone. Subsequently, a depth probe may be used to measure the size of the first hole as well as the appropriate depth to dilate the bone. Those having ordinary skill in the art will appreciate that the depth probe may be device or apparatus known in the art that may be used to measure the size of the first hole formed in the bone and/or the depth of the first hole formed in the bone. Further, referring to FIGS. 1A and 1B, the dilator apparatus 100 may be oriented within the first hole formed in the bone. For example, after the first distal nose 103 has been inserted into the first hole, the longitudinal axis 150 of the dilator apparatus 100 may be substantially aligned with a central axis of the first hole. Further, the dilator apparatus 100 may be twisted, or oriented, about the longitudinal axis 150 to a desired position before further advancing the dilator apparatus 100 into the first hole.

Further, the method may also include forming a second hole within the bone, inserting a second passing pin through the bone, in which the dilation member of the dilator apparatus includes a first distal nose and a second distal nose, and aligning the first distal nose of the dilator apparatus with the first hole and the second distal nose of the dilator apparatus with the second hole.

For example, according to one or more aspects, the first passing pin and the second passing pin may be 2.4 mm passing pins, i.e., the first passing pin and the second passing pin may both be 2.4 mm in diameter. However, those having ordinary skill in the art will appreciate that passing pins of any size may be used. For example, both the first passing pin and the second passing pin may be smaller or lager than 2.4 mm in diameter.

Further, as discussed above with regard to FIGS. 2A and 2B, the dilation member 202 of the dilator apparatus 200 may have a dilation body 209 and a first distal nose 203A coupled to the dilation body 209 and a second distal nose 203B coupled to the dilation body 209, in which a width of the dilation body 209 is larger than both a width of the first distal nose 203A and a width of the second distal nose 203B. According to one or more aspects, a surgeon may place a drill guide (not shown) in an appropriate position on a surface of the bone, e.g., on the footprint of the femur, and may drill two 2.4 mm passing pins through the femur and may form a first hole and a second hole within the bone. The surgeon may then measure the depth of each of the first hole and the second hole. Subsequently, the surgeon may use the appropriate size drill and may drill over the first hole and the second hole to a predetermined size, i.e. diameter, and a predetermined depth. The first distal nose 203A and the second distal nose 203B may then be aligned with the first hole and the second hole, respectively, and the dilator apparatus 200 may be advanced into the bone. Those having ordinary skill in the art will appreciate that, according to one or more aspects, the first hole may be formed to align with the second distal nose 203B and the second hole may be formed to align with the first distal nose 203A. As the dilator apparatus 200 is advanced into the bone, portions of the bone may be removed by the dilator apparatus 200, and the first hole and the second hole may form a single, oblong, e.g., oval-shaped hole. For example, portions of the bone may be removed by the dilation body 209 of the dilator apparatus 200, as the width of the dilation body 209 may be larger than a width the distal noses 203A, 203B, as discussed above. Further, portions of bone between the first hole and the second hole may be displaced by the dilation member 209, as the dilation member 209 extends between the first distal nose 203A and the second distal nose 203B, as shown in FIG. 2B.

Advantageously, embodiments disclosed herein may provide dilator assembly for effectively dilating a hole formed in a bone within a patient's body. For example, as the dilator assembly is advanced into a hole formed in a bone, the tapered region between the first distal nose and the dilation body may allow the diameter of the hole to transition from the diameter of the first distal nose to the diameter of the dilation body. Further, a plurality of broaches formed on an outer surface of the dilation body may assist with bone removal and may assist with the dilation of the hole formed in the bone, e.g. may assist with increasing the diameter and/or shape of the hole formed in the bone. Furthermore, one or more windows formed through the dilator apparatus, according to embodiments disclosed herein, may provide a surgeon which may promote increased visibility, sightline, and viewing as the dilator apparatus is advanced into the bone. These windows formed through the dilator apparatus may also reduce the weight of the dilator apparatus, without sacrificing much torsional stability, which may make bone dilation easier and more effective.

once the adjustable cannula is disposed and effectively engaged within a tissue, as described above, various medical instruments may be inserted and removed from a patient's body while minimizing tissue trauma in the area of skin surrounding the adjustable cannula. Additionally, adjustable cannula, according to embodiments disclosed herein, may provide a fluid seal between the body and the instrument path as well as between the inside and the outside of the body. For example, a first protruding member and a second protruding member, as described above, may form a seal between an exterior tissue surface and an interior tissue surface, respectively. Specifically, the seal formed by the first protruding member and the second protruding member may prevent fluid from escaping from the body during a surgical procedure. Likewise, the seal formed by the first protruding member and the second protruding member may prevent fluid from entering the body during a surgical procedure.

The adjustable cannula, according to embodiments disclosed herein, may also allow a surgeon to adjust the length of the tubular body of the adjustable cannula to accommodate for the tissue thickness of a particular patient. The adjustability of the length of the adjustable cannula may allow surgeons to use the cannula with a variety of patients, varying in size. Further, the adjustability of the length of the adjustable cannula may allow surgeons to use the cannula to access a variety of areas of interest within the body, e.g., various joints as well as various regions of the pelvic area. For example, the adjustable cannula may allow surgeons to use the cannula to access joints, such as a hip or shoulder joint, as well as a variety of sealed organs, such as a kidney or liver. Furthermore, as discussed above, engaging the second protruding member of the tubular body of the adjustable cannula may secure the adjustable cannula to the interior tissue surface and prevent undesired removal of the adjustable cannula during a surgical procedure. Additionally, the first protruding member and the second protruding member of the adjustable cannula may also provide a surface for tissue, e.g., subcutaneous tissue, to compress against. Compressing tissue with the first protruding member and the second protruding member of the adjustable cannula may shorten the pathway that medical instruments may have to travel in order to access areas of interest within the body. Finally, according to embodiments disclosed herein, once the second protruding member is engaged with the interior tissue surface of a body, the adjustable cannula may be used, e.g., pulled, to manipulate the interior tissue of a body, e.g., a joint, instrument workability, and work space within the body.

While embodiments have been described with respect to a limited number of embodiments, those skilled in the art, having benefit of this disclosure, will appreciate that other embodiments can be devised which do not depart from the scope of embodiments disclosed herein. Accordingly, the scope of embodiments disclosed herein should be limited only by the attached claims.

What is claimed is:

1. A dilator apparatus comprising:
a body having a proximal end and a distal end and a longitudinal axis defined therethrough; and
a dilation member coupled to the distal end of the body, the dilation member comprising a dilation body and a first distal nose coupled to the dilation body,
wherein a width of the dilation body is larger than a width of the first distal nose.

2. The apparatus of claim 1, wherein the width of the dilation body is larger than a width of the body.

3. The apparatus of claim 1, wherein a cross-section of the body is substantially oval-shaped.

4. The apparatus of claim 1, wherein at least one window is formed through the body.

5. The apparatus of claim 1, wherein the dilation member comprises a second distal nose coupled to the dilation body.

6. The apparatus of claim 5, wherein the width of the dilation body is larger than a width of the second distal nose.

7. The apparatus of claim 1, wherein a region between the first distal nose and the dilation body is a tapered region.

8. The apparatus of claim 5, wherein a region between the second distal nose and the dilation body is a tapered region.

9. The apparatus of claim 1, wherein the dilation body comprises a plurality of broaches.

10. The apparatus of claim 1, further comprising a handle coupled to the proximal end of the body.

11. The apparatus of claim 1, wherein a hole is formed through the body and the dilation member along the longitudinal axis of the body.

12. A method for dilating a bone, the method comprising:
forming a first hole within a bone;
inserting a dilator apparatus into the first hole, the dilator apparatus comprising a body and a dilation member, wherein the dilation member is coupled to a distal end of the body; and
advancing the dilator apparatus within the first hole formed in the bone to a predetermined depth within the bone.

13. The method of claim 12, further comprising inserting a first passing pin through the bone.

14. The method of claim 12, further comprising measuring a size of the first hole and the predetermined depth within the bone with a depth probe.

15. The method of claim 12, further comprising orienting the dilator apparatus within the first hole formed in the bone.

16. The method of claim 12, wherein advancing the dilator apparatus within the first hole formed in the bone comprises advancing the dilator apparatus within the first hole formed in the bone with a mallet.

17. The method of claim 12, further comprising forming a second hole within the bone.

18. The method of claim 17, further comprising inserting a second passing pin through the bone.

19. The method of claim 17, wherein the dilation member of the dilator apparatus comprises a first distal nose and a second distal nose.

20. The method of claim 19, further comprising aligning the first distal nose of the dilator apparatus with the first hole and the second distal nose of the dilator apparatus with the second hole.
